(19) United States
(12) Patent Application Publication
(10) Pub. No.: US 2022/0047857 A1
(43) Pub. Date: Feb. 17, 2022

(54) MANUFACTURING METHOD FOR MICRO-NEEDLE DEVICE

(71) Applicant: Tamkang University, New Taipei City (TW)

(72) Inventors: Ming-Kai Chern, New Taipei City (TW); Man-Piu Chan, New Taipei City (TW); Yueh-Tzu Lo, New Taipei City (TW); I-Chang Liu, New Taipei City (TW); Shih-Ting Lin, New Taipei City (TW); You-Lin Wei, New Taipei City (TW); Wen-Chi Chou, New Taipei City (TW); Wen-Hua Chuang, New Taipei City (TW); Yin-Jun Wu, New Taipei City (TW); Hun-Boa Wang, New Taipei City (TW); Bo-Cheng Wang, New Taipei City (TW)

(73) Assignee: Tamkang University, New Taipei City (TW)

(21) Appl. No.: 17/085,427

(22) Filed: Oct. 30, 2020

(30) Foreign Application Priority Data
Aug. 12, 2020 (TW) 109127435

(51) Int. Cl. A61M 37/00; B29C 45/26; B81C 1/00

(52) U.S. Cl. CPC A61M 37/0015; B29C 45/26; B29L 2031/7544; A61M 2037/0061; B81C 1/00111

(57) ABSTRACT

A manufacturing method for a micro-needle device includes following steps: a target tissue basic information obtaining step, a micro-needle template obtaining step, a micro-needle material adding step, a micro-needle semi-product obtaining step, and a micro-needle device obtaining step. The inner tissue distribution information is obtained by the application of optical coherence tomography. The micro-needle template is obtained according to the skin surface curvature information and the inner tissue distribution information. The micro-needle template has a plurality of areas and a plurality of mold holes. One or both of the diameter and the depth of the mold hole is determined by the inner tissue distribution information, and the curvature radius of the areas is determined by the skin surface curvature information. The manufacturing method for a micro-needle device is applicable to micro-needles with mixed configurations as well as micro-needles with syringe configurations.

### CROSS-REFERENCE TO RELATED APPLICATION

This non-provisional application claims priority under 35 U.S.C. § 119(a) to Patent Application No. 109127435 filed in Taiwan, R.O.C. on Aug. 12, 2020, the entire contents of which are hereby incorporated by reference.

### BACKGROUND

### Technical Field

The instant disclosure relates to a micro-needle, in particular to a manufacturing method for a micro-needle device.

### Related Art

Oral administration is a common way for supplying medicine to a human. However, due to the liver primary metabolism or dyspepsia, the time for medicine absorption may be extended and the medical effect may be worsened. Intravenous injection or other subcutaneous injection methods may be used for delivering substances into the blood. However, professional or trained personnel are required for the operation. Otherwise, several adverse reactions may occur.

Micro-needle is a new-generation transderaml drug delivery system (TDDS). The micro-needle can deliver active substances to subcutaneous tissues or bloods with certain rates in an effective manner to reduce absorption variability of substances and to maintain the concentration of the active substances in the bloods. Furthermore, micro-needle treatments are painless therapeutical procedures, so that the users are more willing to have the treatments.

### SUMMARY

Generally speaking, micro-needles can be further classified into an insoluble micro-needle and a soluble micro-needle based on whether a micro-needle body is absorbable (for example, being biodegradable and water-soluble materials) by the human body. In a manufacturing process of a soluble micro-needle, polydimethylsiloxane (PDMS) is generally used for rolling over a mold. However, the process in high in molding cost. Furthermore, in a traditional micro-needle, a needle body of the micro-needle is configured on a horizontal plane, and the size and shape of the needle are also fixed, and cannot be adjusted for different users, such that the application of the micro-needle cannot achieve the optimal effect.

In view of this, one or more embodiments of the instant disclosure provide a manufacturing method for a micro-needle device, which includes a target tissue basic information obtaining step, a micro-needle template obtaining step, a micro-needle material adding step, a micro-needle semi-product obtaining step, and a micro-needle device obtaining step. In the target tissue basic information obtaining step, skin surface curvature information and inner tissue distribution information of a target tissue are obtained. The inner tissue distribution information is obtained by applying optical coherence tomography. In the micro-needle template obtaining step, a micro-needle template is obtained according to the skin surface curvature information and the inner tissue distribution information. The micro-needle template has a plurality of areas and a plurality of mold holes, at least one of the plurality of mold holes is located in at least one of the plurality of areas, at least one of the diameter and the depth of the plurality of mold holes is determined by the inner tissue distribution information, and the curvature radius of the plurality of areas is determined by the skin surface curvature information. In the micro-needle material adding step, a micro-needle material is added to the micro-needle template, such that the micro-needle material is located on the plurality of areas and fills the mold holes. The micro-needle material includes a molding substance. In the micro-needle semi-product obtaining step, the micro-needle material is solidified to form a micro-needle semi-product. In the micro-needle device obtaining step, the micro-needle template is removed to obtain the micro-needle device.

In one or more embodiments, the micro-needle semi-product obtaining step is performed under a temperature ranging from 0° C. to −196° C. In some embodiments, the above micro-needle semi-product obtaining step may be performed in a cyclic manner. That is, the micro-needle material is solidified through a freezing cycle to obtain the micro-needle semi-product. Furthermore, the micro-needle material further includes an active substance, and the micro-needle device obtaining step is to solidify the micro-needle semi-product under a temperature ranging from 50° C. to 90° C. to obtain the micro-needle device.

In one or more embodiments, the micro-needle semi-product obtaining step is performed under a temperature ranging from 50° C. to 90° C. In some embodiments, the above micro-needle semi-product obtaining step may be performed by setting the temperature at a fixed value. That is, the micro-needle material is solidified by utilizing a constant temperature environment to obtain the micro-needle semi-product. Furthermore, in some embodiments, the obtained micro-needle device has a groove for containing the active substance.

In one or more embodiments, the molding substance is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan.

In one or more embodiments, the micro-needle semi-product obtaining step is performed under a room temperature, the micro-needle material further includes an active substance, and the molding substance is collagen or hyaluronic acid. Furthermore, the micro-needle device obtaining step is to solidify the micro-needle semi-product under a temperature ranging from 50° C. to 90° C. to obtain the micro-needle device.

In one or more embodiments, before the micro-needle material adding step, the method further includes a template protection layer forming step: forming a template protection layer on the micro-needle template under a temperature ranging from 50° C. to 90° C., such that the template protection layer is located on the plurality of areas and fills the plurality of mold holes. The micro-needle material is located on the plurality of areas and on the template protection layer, the template protection layer is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan, and the micro-needle material further comprises the active substance. Furthermore, the micro-needle device obtaining step is to remove the micro-needle template and the template protection layer to obtain the micro-needle device.

In one or more embodiments, the micro-needle semi-product obtaining step is performed under a room temperature or under a temperature ranging from 0° C. to −196° C. Furthermore, the micro-needle device obtaining step is to solidify the micro-needle semi-product under a temperature ranging from 50° C. to 90° C. to obtain the micro-needle device.

In one or more embodiments, the micro-needle semi-product obtaining step is performed under a temperature ranging from 0° C. to −196° C. or a temperature ranging from 50° C. to 90° C.

In one or more embodiments, the micro-needle semi-product obtaining step is performed under a temperature ranging from 50° C. to 90° C., and the molding substance is collagen or hyaluronic acid. The micro-needle device obtaining step is to solidify the micro-needle semi-product under a temperature ranging from 50° C. to 90° C. to obtain the micro-needle device.

In one or more embodiments, the micro-needle semi-product obtaining step is performed under a temperature ranging from 50° C. to 90° C., and the molding substance is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan. The micro-needle device obtaining step is to solidify the micro-needle semi-product under a temperature ranging from 0° C. to −196° C. or from 50° C. to 90° C. to obtain the micro-needle device.

In one or more embodiments, the template protection layer forming step further includes: immersing the micro-needle template in a protection layer solution; heating the micro-needle template and the protection layer solution to a temperature ranging from 50° C. to 90° C. to form the template protection layer on the micro-needle template; and taking the micro-needle template with the template protection layer out of the protection layer solution.

In one or more embodiments, the template protection layer forming step further includes: adding a solvent to the micro-needle template; immersing the micro-needle template in a protection solution tank, wherein the protection solution tank contains the protection layer solution; mixing the solvent and the protection layer solution; heating the protection solution tank to a temperature ranging from 50° C. to 90° C. to form the template protection layer on the micro-needle template; and taking the micro-needle template with the template protection layer out of the protection solution tank.

In one or more embodiments, the skin surface curvature information of the target tissue is obtained by utilizing a three-dimensional scanning technology or the optical coherence tomography. Further, in some embodiments, the template protection layer forming step further includes: obtaining a micro-injector array by utilizing the three-dimensional scanning technology or the optical coherence tomography, wherein the micro-injector array has a container and a plurality of injection needles, each injection needle has a needle hole for communicating with the container, and the size of the plurality of injection needles corresponds to the diameter and depth of the plurality of mold holes; providing the protection layer solution into the container, and enabling the protection layer solution to pass through the needle holes, be located in the areas and enter into the plurality of mold holes; taking the micro-injector array out; heating the micro-needle template and the protection layer solution to a temperature ranging from 50° C. to 90° C. to form a micro-needle protection layer on the micro-needle template; and taking the micro-needle template out of the protection layer solution.

Another embodiment of the instant disclosure discloses a manufacturing method for a micro-needle device, including: a target tissue basic information obtaining step, a first micro-needle template obtaining step, a template protection layer forming step, a micro-needle material adding step, a second micro-needle template obtaining step, a second micro-needle template configuring step, a micro-needle material solidifying step, a second micro-needle template removing step, an active substance adding step, and a micro-needle device obtaining step. In the target tissue basic information obtaining step, skin surface curvature information and inner tissue distribution information of a target tissue is obtained. The inner tissue distribution information is obtained by applying optical coherence tomography. In the first micro-needle template obtaining step, a first micro-needle template is obtained according to the skin surface curvature information and the inner tissue distribution information. The first micro-needle template has a plurality of first areas and a plurality of mold holes, at least one of the plurality of mold holes is located in at least one of the plurality of first areas, at least one of the diameter and the depth of the plurality of mold holes is determined by the inner tissue distribution information, and the curvature radius of the plurality of first areas is determined by the skin surface curvature information. In a template protection layer forming step, a template protection layer is formed on the first micro-needle template, such that the template protection layer is located on the plurality of first areas and fills the plurality of mold holes. In a micro-needle material adding step, a micro-needle material is added to the template protection layer, such that the micro-needle material is located on the plurality of first areas and fills the plurality of mold holes. The micro-needle material includes a molding substance. In the second micro-needle template obtaining step, a second micro-needle template is obtained according to the skin surface area information and the inner tissue distribution information. The second micro-needle template has a plurality of second areas and a plurality of needle-shaped structures, at least one of the plurality of needle-shaped structures is located in at least one of the plurality of second areas, the diameter and the length of the plurality of needle-shaped structures correspond to the diameter and the depth of the plurality of mold holes respectively, and the curvature radius of the plurality of second areas corresponds to the curvature radius of the plurality of first areas. In the second micro-needle template configuring step, the second micro-needle template is configured on the micro-needle material and the first micro-needle template, such that the plurality of second areas are located on the plurality of first areas correspondingly, and the needle-shaped structures are inserted into the plurality of mold holes correspondingly, and the micro-needle material is located between the first micro-needle template and the second micro-needle template. In the micro-needle material solidifying step, the micro-needle material is solidified to form a micro-needle semi-product. The micro-needle semi-product includes a plurality of micro-needle bodies, and each micro-needle body has a hole. In the second micro-needle template removing step, the second micro-needle template is removed to keep the micro-needle semi-product and the first micro-needle template left. In the active substance adding step, an active substance is added to the micro-needle semi-product, and the active substance is enabled to enter into the holes. In the micro-needle device obtaining step, the first micro-needle template is removed, and the micro-needle semi-product is solidified to obtain a micro-needle device.

In one or more embodiments, the template protection layer forming step further includes: immersing the first micro-needle template in a protection layer solution; heating the first micro-needle template and the protection layer solution to a temperature ranging from 50° C. to 90° C. to form the template protection layer on the first micro-needle template; and taking the first micro-needle template with the template protection layer out of the protection layer solution.

In one or more embodiments, the template protection layer forming step further includes: adding a solvent to the first micro-needle template; immersing the first micro-needle template in a protection solution tank, where the protection solution tank contains the protection layer solution; mixing the solvent and the protection layer solution; heating the protection solution tank to a temperature ranging from 50° C. to 90° C. to form the template protection layer on the first micro-needle template; and taking the first micro-needle template with the template protection layer out of the protection solution tank.

In one or more embodiments, the skin surface curvature information of the target tissue is obtained by utilizing a three-dimensional scanning technology or the optical coherence tomography. Further, in one or more embodiments, the template protection layer forming step further includes: obtaining a micro-injector array by utilizing the three-dimensional scanning technology or the optical coherence tomography, where the micro-injector array has a container and a plurality of injection needles, each injection needle has a needle hole for communicating with the container, and the size of the plurality of injection needles corresponds to the diameter and depth of the plurality of mold holes; providing the protection layer solution into the container, and enabling the protection layer solution to pass through the plurality of needle holes, be located in the plurality of first areas and enter into the plurality of mold holes; taking the micro-injector array out; heating the first micro-needle template and the protection layer solution to a temperature ranging from 50° C. to 90° C. to form a first micro-needle protection layer on the first micro-needle template; and taking the first micro-needle template out of the protection layer solution.

In some embodiments, the template protection layer may be made in a physical or chemical mode. Specifically, in terms of the physical mode, for example, the template protection layer may be made by irradiating a light-harden material with ultraviolet rays or changing the form of a specific material through temperature changes; and on the other hand, in terms of the chemical mode, the template protection layer may be made with a polymer in cooperation with an appropriate cross-linking agent.

In some embodiments, a corresponding skin model can be made by using the three-dimensional printing technology based on the foregoing information, and then the micro-needle template can be made with the skin model as a basic structure, but it is not limited to this; in some embodiments, the micro-needle template can be made directly by using the three-dimensional printing technology based on the foregoing information.

In summary, according to one or more embodiments of the instant disclosure, a micro-needle device with a syringe or mixed type needle body can be manufactured according to different usage requirements, and a high-specificity micro-needle device product can be made corresponding to specific skin surface curvature information and inner tissue distribution information of a user. In addition, in some embodiments, the skin condition of the user may also be understood according to the inner tissue distribution information, then in the active substance adding step, different positions of the micro-needle device have different contents of active substances, so that the provision efficiency of the active substance is optimized. In still other embodiments, air bubbles can be reduced during molding, thereby ensuring the integrity of the protection layer/the micro-needle device.

### BRIEF DESCRIPTION OF THE DRAWINGS

The disclosure will become more fully understood from the detailed description given herein below for illustration only, and thus not limitative of the disclosure, wherein:

FIG. 1 illustrates a step flowchart of a manufacturing method for a micro-needle device of Embodiment 1 of the instant disclosure;

FIG. 2 illustrates a perspective schematic view of a micro-needle template of one embodiment of the instant disclosure;

FIG. 3A to FIG. 3D illustrate schematic cross-sectional views corresponding to different steps of the manufacturing method for the micro-needle device of Embodiment 1 of the instant disclosure;

FIG. 4 illustrates a step flowchart of a manufacturing method for a micro-needle device of Embodiment 2 of the instant disclosure;

FIG. 5 illustrates a schematic cross-sectional view of a micro-needle template configured with a template protection layer of one embodiment of the instant disclosure;

FIG. 6 illustrates a step flowchart of a manufacturing method for a micro-needle device of Embodiment 3 of the instant disclosure;

FIG. 7A illustrates a perspective schematic view of a first micro-needle template of one embodiment of the instant disclosure;

FIG. 7B illustrates a perspective schematic view of a second micro-needle template of one embodiment of the instant disclosure;

FIG. 8A to FIG. 8D illustrate schematic cross-sectional views corresponding to different steps of the manufacturing method for the micro-needle device of Embodiment 3 of the instant disclosure;

FIG. 9 illustrates a partial step flowchart of a manufacturing method for a micro-needle device of Embodiment 4 of the instant disclosure;

FIG. 10 illustrates a partial step flowchart of a manufacturing method for a micro-needle device of Embodiment 5 of the instant disclosure;

FIG. 11 illustrates a detailed flowchart of a template protection layer forming step of one embodiment of the instant disclosure;

FIG. 12 illustrates a detailed flowchart of a template protection layer forming step of another embodiment of the instant disclosure;

FIG. 13 illustrates a detailed flowchart of a template protection layer forming step of yet another embodiment of the instant disclosure; and

FIG. 14 illustrates a schematic cross-sectional view of a micro-needle template matched with a micro-injector array corresponding to the embodiment shown by FIG. 13.

### DETAILED DESCRIPTION

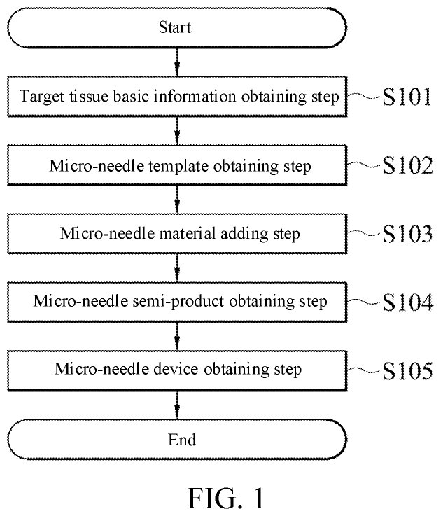

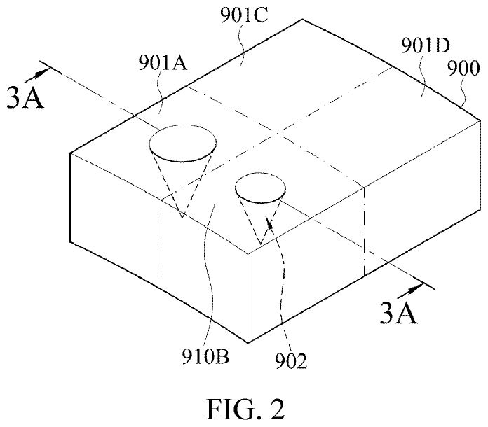

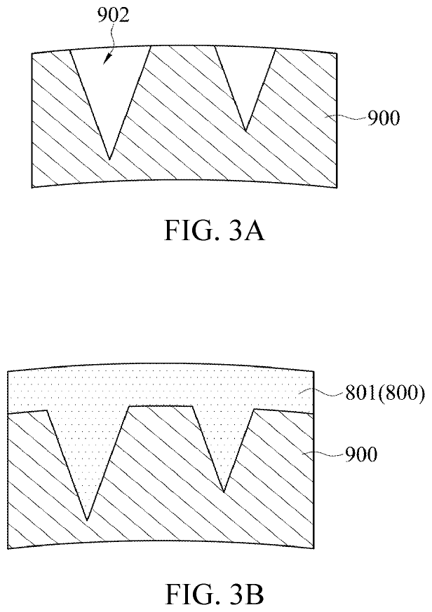

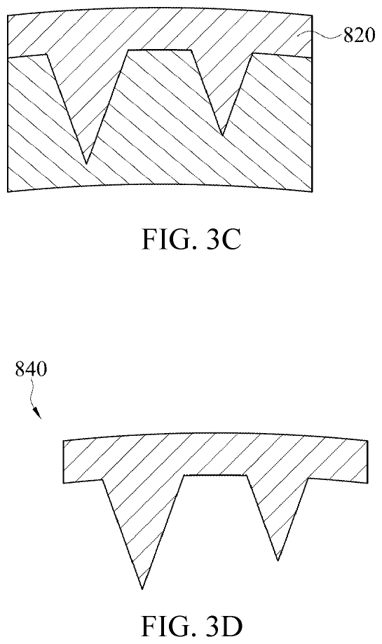

Referring to FIG. 1, FIG. 2, and FIGS. 3A to 3D, FIG. 1 illustrates a step flowchart of a manufacturing method for a micro-needle device of Embodiment 1 of the instant disclosure, FIG. 2 illustrates a perspective schematic view of a micro-needle template of one embodiment of the instant disclosure, and FIGS. 3A to 3D illustrate schematic cross-sectional views corresponding to different steps of the manufacturing method for the micro-needle device of Embodiment 1 of the instant disclosure. As shown in the figures, the manufacturing method for the micro-needle device includes the following steps: a target tissue basic information obtaining step S101, a micro-needle template obtaining step S102, a micro-needle material adding step S103, a micro-needle semi-product obtaining step S104, and a micro-needle device obtaining step S105.

In this embodiment, a mixed type micro-needle or a syringe type micro-needle can be made according to requirements. Specifically, in one or some embodiments, if a needle body of the micro-needle device further contains, in addition to a molding material, an active substance (such as a beauty formula (such as hyaluronic acid, collagen, etc.), a pharmaceutical composition (such as a natural extract, a compound ingredient, etc.), a macromolecular medicine (such as a vaccine, an antibody, insulin, etc.), and a small molecule medicine (such as anesthetics, an anti-cancer medicine, etc.), such that the active substance can be absorbed by a target tissue after being applied to a skin surface of the target tissue (such as human skin), then the device can be defined as the mixed type micro-needle. If a needle body of the micro-needle device only has a molding material, then an active substance is applied to a hole formed in the needle body through a subsequent process. In this way, when the needle body is absorbed by a target tissue to a certain extent, the active substance in the hole can be released and absorbed by the target tissue. In this case, the device is defined as the syringe type micro-needle.

In the target tissue basic information obtaining step S101, skin surface curvature information of the target tissue and inner tissue distribution information of the target tissue are obtained. Herein, a structural state of the skin surface of the target tissue is analyzed (for example, the target tissue is located at a joint, so that skin surface curvature of the same area has a certain amount of change) to obtain the skin surface curvature information of the target tissue. Furthermore, in this embodiment, the skin surface curvature information of the target tissue is obtained by utilizing a three-dimensional scanning technology. On the other hand, a distribution condition of an inner tissue of the target tissue (such as the thickness of an epidermal layer/a dermis layer, and distribution positions and depths of a blood vessel, lymph, and a connective tissue of the target tissue) is analyzed, and the inner tissue distribution information is obtained by utilizing optical coherence tomography.

In one or more embodiments, the skin surface curvature information of the target tissue is obtained by utilizing the three-dimensional scanning technology or the optical coherence tomography.

Optical coherence tomography (hereinafter referred to as OCT) is a method for obtaining and processing optical signals. It utilizes a principle of light interference to scan an optical scattering medium (such as the target tissue) to obtain longitudinal profile data and transverse profile data through the reflection of light by the target tissue instead of devastatingly providing a cross-sectional image of the target tissue, and further obtain inner tissue distribution information based on the longitudinal profile data and the transverse profile data.

Next, in the micro-needle template obtaining step S102, the micro-needle template 900 is obtained based on the skin surface curvature information and the inner tissue distribution information. In this embodiment, a corresponding skin model can be made by using the three-dimensional printing technology based on the foregoing information, and then the micro-needle template 900 can be made with the skin model as a basic structure, but it is not limited to this. In some embodiments, the micro-needle template 900 can be made directly by using the three-dimensional printing technology based on the foregoing information. Referring to FIG. 2 and FIG. 3A, the micro-needle template 900 has a plurality of areas 901A, 901B, 901C, 901D and a plurality of mold holes 902, and at least one of the plurality of mold holes 902 is located in at least one of the plurality of areas. In other words, as shown in FIG. 2, in this embodiment, the micro-needle template 900 has the plurality of areas 901A, 901B, 901C, and 901D, The areas 901A and 901B are configured with the plurality of mold holes 902 according to the foregoing information of the target tissue, while the area 901C and area 901D are not configured with the plurality of mold holes 902 due to the existence of the blood vessel/the lymph/the connective tissue/a nerve at the corresponding portion(s) of the target tissue. In addition, at least one of the diameter and the depth of the plurality of mold holes 902 is determined by the inner tissue distribution information, and the curvature radius of the areas 901A, 901B, 901C and 901D is determined by the skin surface curvature information. In other words, the diameter and/or the depth of the mold hole 902 can be determined according to the location and range of the blood vessel, the lymph, and the connective tissue provided by the inner tissue distribution information. A curvature radius of the micro-needle template 900 is determined by a contour change of the skin surface corresponding to the target tissue, the curvature radius of the micro-needle template 900 in the figure is only illustrative, and it is not limited to this.

Next, as shown in FIG. 3B, in the micro-needle material adding step S103, a micro-needle material 800 is added to the micro-needle template 900, such that the micro-needle material 800 is located on the plurality of areas 901A, 901B, 901C and 901D and fills the plurality of mold holes 902. The micro-needle material 800 includes a molding substance 801. In one or more embodiments, the molding substance 801 is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan. In this embodiment, the molding substance 801 is an appropriate biodegradable material, and therefore can be directly applied to a target tissue of a user and absorbed and decomposed.

Second, as shown in FIG. 3C, in the micro-needle semi-product obtaining step S104, the micro-needle material 800 is solidified to form a micro-needle semi-product 820. In some embodiments, the micro-needle semi-product obtaining step S104 is performed under a temperature ranging from 50° C. to 90° C. Further, in some embodiments, the micro-needle material 800 is solidified into the semi-finished micro-needle 820 in a constant temperature heating mode in the above temperature range. Alternatively, in some embodiments, the micro-needle semi-product obtaining step S104 is performed under a temperature ranging from 0° C. to −196° C. Further, in some embodiments, the micro-needle material 800 is solidified into the semi-finished micro-needle 820 in a freezing cycle drying mode in the above temperature range. It should be noted that the temperature mentioned in one or more embodiments of the instant disclosure refers to a set temperature of a processing environment.

Then, as shown in FIG. 3D, in the micro-needle device obtaining step S105, the micro-needle template 900 is removed to obtain a micro-needle device 840. When the foregoing micro-needle semi-product obtaining step S104 is performed under a temperature ranging from 50° C. to 90° C., a groove is formed in the obtained micro-needle device 840 due to a heating solidifying mode. In some embodiments, after the micro-needle device 840 is made, a hole can be formed in each needle body on the micro-needle device 840 through a precise machining mode or a micro-electromechanical systems (MEMS) machining, and the like, and the active substance can be firstly mixed with an excipient and a stabilizer and then fills the hole. In this way, a user can fill an appropriate quantity of active substances into the groove or the hole to make a micro-needle product or a micro-needle patch that can transfer the active substance. In some embodiments, in addition to the excipient and the stabilizer, the active substance may also be mixed with a macromolecular material that can form micelles, thereby protecting the active substance and even controlling release of the active substance through the micelles.

In some embodiments, as shown in FIG. 3D, the needle bodies on the micro-needle device 840 may be arranged in parallel. In this way, when the micro-needle device 840 is subsequently applied to skin of the user, a moment of lateral force received by each needle body is equalized without generating resistance, and when the micro-needle device 840 is applied to the micro-needle patch product, it is less likely to be deformed. In some other embodiments, the needle bodies are not arranged in parallel, but in a configuration in which the needle bodies extend in a normal direction of the surface of the micro-needle device 840.

In some embodiments, the skin condition of the user may also be understood according to the inner tissue distribution information, then in the active substance adding step, different positions of the micro-needle device 840 have different contents of active substances (for example, the content of an active substance of a first needle body of the micro-needle device is less than that of other needle bodies), so that an appropriate active substance can be provided to an application position of the user more efficiently.

The needle body on the micro-needle device 840 may also be a mixed type micro-needle in addition of a syringe type micro-needle. In one or more embodiments, the micro-needle material 800 further includes the active substance. That is, the micro-needle material 800 is a mixture of the molding substance 801 and the active substance, so that a needle body on the micro-needle device 840 produced subsequently has the molding substance 801 and the active substance. Therefore, when the needle body of the micro-needle device 840 is inserted into the target tissue, the active substance can be quickly absorbed.

When a needle body used is a mixed type micro-needle, there may be the following parameter configuration. In some embodiments, the micro-needle semi-product obtaining step S104 is performed under a temperature ranging from 0° C. to −196° C. Moreover, in some embodiments, the step can be performed in a freezing cycle mode so as to solidify the micro-needle material 800. In some embodiments, the micro-needle semi-product obtaining step S104 is performed under a room temperature, and the molding substance 801 is collagen. Under the foregoing state, the micro-needle device obtaining step S105 is to solidify the micro-needle semi-product S820 under a temperature ranging from 50° C. to 90° C. to obtain the micro-needle device 840.

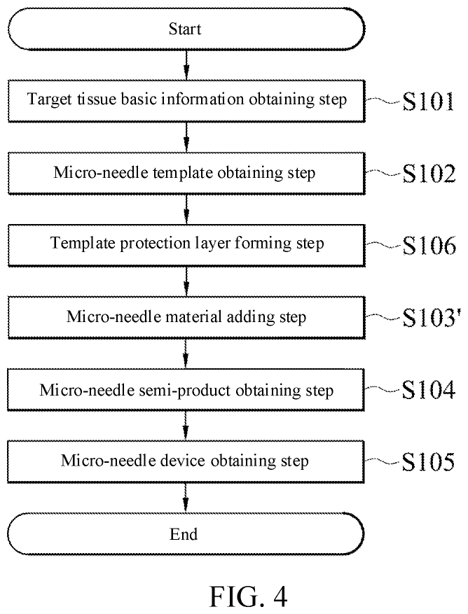

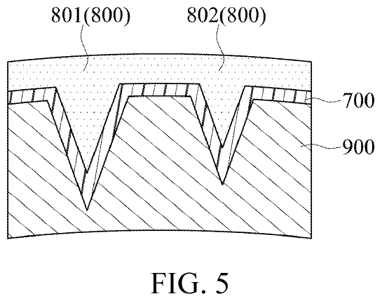

Referring to FIG. 4, FIG. 5 and FIG. 1, FIG. 4 illustrates a step flowchart of a manufacturing method for a micro-needle device of Embodiment 2 of the instant disclosure, and FIG. 5 illustrates a schematic cross-sectional view of a micro-needle template configured with a template protection layer of one embodiment of the instant disclosure. In this embodiment, before a micro-needle material adding step S103′, a template protection layer forming step S106 is further included: a template protection layer 700 is formed on a micro-needle template 900 under a temperature ranging from 50° C. to 90° C., such that the template protection layer 700 is located on the plurality of areas 901A, 901B, 901C and 901D and fills the plurality of mold holes 902, as shown in FIG. 5. Therefore, in the micro-needle material adding step S103′, the micro-needle material 800 is located on the plurality of areas 901A, 901B, 901C, 901D and on the template protection layer 700.

In this embodiment, the template protection layer 700 is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan, but it is not limit to this. In other words, in this embodiment, a molding substance 801 and the template protection layer 700 may be made from the same material, but may also be made from different materials. The template protection layer 700 is used to separate the micro-needle material 800 from the micro-needle template 900, thereby facilitating a subsequent demolding step after the micro-needle material 800 is molded. In some embodiments, the template protection layer 700 may be made in a physical or chemical mode. Specifically, in terms of the physical mode, for example, the template protection layer 700 may be made by irradiating a light-harden material with ultraviolet rays or changing the form of a specific material through temperature changes; and on the other hand, in terms of the chemical mode, the template protection layer 700 may be made with a polymer in cooperation with an appropriate cross-linking agent.

In this embodiment, a needle body of the made micro-needle device 840 is a mixed type micro-needle. In other words, in this embodiment, the micro-needle material 800 includes the molding substance 801 and an active substance 802.

In this embodiment, in a micro-needle device obtaining step S105′, the micro-needle template 900 and the template protection layer 700 are removed to obtain the micro-needle device 840.

In one or more embodiments, a micro-needle semi-product obtaining step S104 is performed under a room temperature or under a temperature ranging from 0° C. to −196° C. Moreover, in the micro-needle device obtaining step S105′, a micro-needle semi-product is solidified under a temperature ranging from 50° C. to 90° C. to obtain the micro-needle device 840.

In one or more embodiments, the micro-needle semi-product obtaining step S104 is performed under a temperature ranging from 50° C. to 90° C., and the molding substance is collagen or hyaluronic acid. In addition, in the micro-needle device obtaining step S105′ is to solidify the micro-needle semi-product 820 under a temperature ranging from 50° C. to 90° C. to obtain the micro-needle device 840. In other words, in this embodiment, the molding substance 801 and the template protection layer 700 are made from different materials.

In one or more embodiments, the micro-needle semi-product obtaining step S104 is performed under a temperature ranging from 50° C. to 90° C., and the molding substance 801 is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan. In other words, in this embodiment, the molding substance 801 and the template protection layer 700 may be made from the same material. In addition, the micro-needle device obtaining step S105′ is to solidify the micro-needle semi-product S820 under a temperature ranging from 0° C. to −196° C. or from 50° C. to 90° C. to obtain the micro-needle device 840.

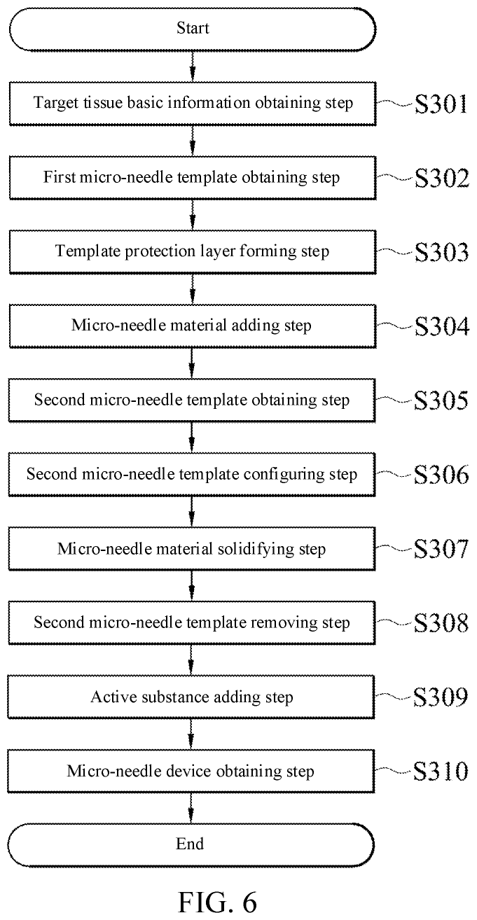

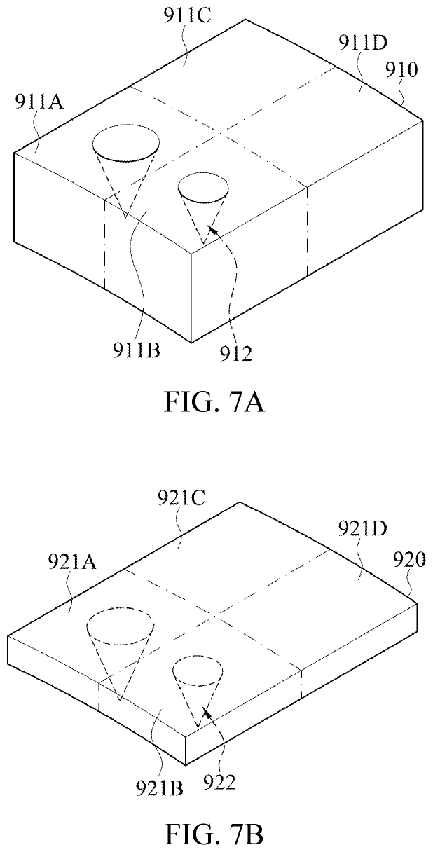

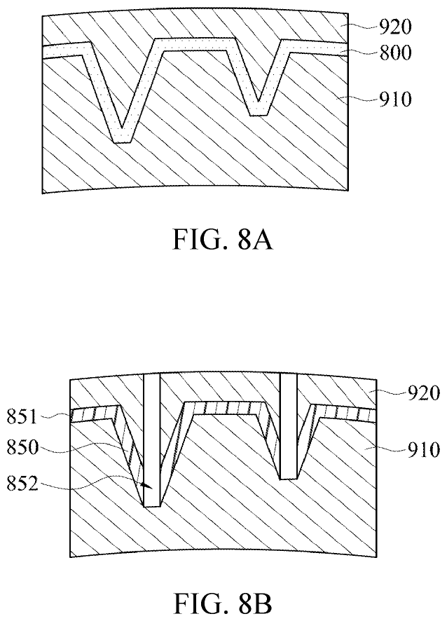

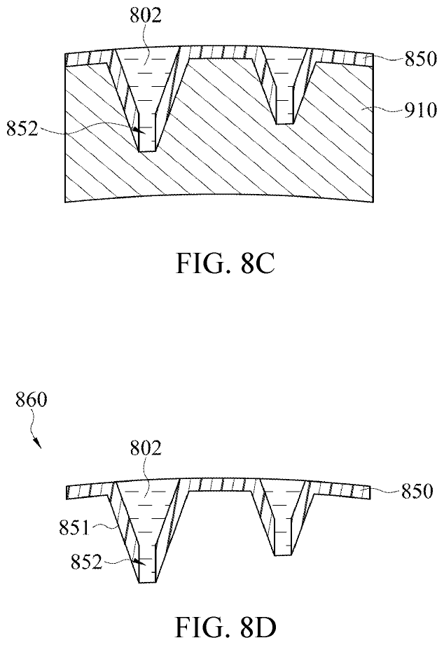

Referring to FIG. 6, FIG. 7A, FIG. 7B and FIGS. 8A to 8D, FIG. 6 illustrates a step flowchart of a manufacturing method for a micro-needle device of Embodiment 3 of the instant disclosure, FIG. 7A and FIG. 7B illustrate a perspective schematic view of a first micro-needle template of one embodiment of the instant disclosure and a perspective schematic view of a second micro-needle template of one embodiment of the instant disclosure, respectively, and FIG. 8A to FIG. 8D are schematic cross-sectional views corresponding to different steps of the manufacturing method for the micro-needle device of Embodiment 3 of the instant disclosure. As shown in the figures, a manufacturing method for a micro-needle device includes the following steps: a target tissue basic information obtaining step S301, a first micro-needle template obtaining step S302, a template protection layer forming step S303, a micro-needle material adding step S304, a second micro-needle template obtaining step S305, a second micro-needle template configuring step S306, a micro-needle material solidifying step S307, a second micro-needle template removing step S308, an active substance adding step S309, and a micro-needle device obtaining step S310.

In the target tissue basic information obtaining step S301, skin surface curvature information of a target tissue and inner tissue distribution information of the target tissue are obtained. As mentioned above, inner tissue distribution information is obtained by applying optical coherence tomography, which will not be repeated any more.

Next, in the first micro-needle template obtaining step S302, a first micro-needle template 910 is obtained based on the skin surface curvature information and the inner tissue distribution information. Referring to FIG. 7A at the same time, the first micro-needle template 910 has a plurality of first areas 911A, 911B, 911C and 911D and a plurality of mold holes 912, at least one of the plurality of mold holes 912 is located in at least one of the plurality of first areas 911A, 911B, 911C and 911D, at least one of the diameter and the depth of the mold holes 912 is determined by the inner tissue distribution information, and the curvature radius of the plurality of first areas 911A, 911B, 911C and 911D is determined by the skin surface curvature information. This step is basically the same as the micro-needle template obtaining step S102, and will not be repeated any more.

Then, in the template protection layer forming step S303, a template protection layer 700 is formed on a first micro-needle template 910, such that the template protection layer 700 is located on the plurality of first areas 911A, 911B, 911C and 911D and fills the plurality of mold holes 912. The manufacturing method, material selection and the like of the template protection layer 700 have been described in the previous paragraphs, and will not be repeated herein any more.

Next, the micro-needle material adding step S304 follows. In the step, a micro-needle material 800 is added to the micro-needle template 700, such that the micro-needle material 800 is located on the plurality of first areas 911A, 911B, 911C and 911D and fills the plurality of mold holes 912. The micro-needle material 800 includes a molding substance 801. In one or more embodiments, the molding substance 801 is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan. In this embodiment, the molding substance 801 is an appropriate biodegradable material, and therefore can be directly applied to a target tissue of a user and absorbed and decomposed.

Then, the second micro-needle template obtaining step S305 follows. In the step, a second micro-needle template 920 is obtained based on the skin surface curvature information and the inner tissue distribution information. Specifically, the second micro-needle template 920 corresponding to a first micro-needle template 910 in structure is made by using a three-dimensional printing technology based on the foregoing information. Referring to FIG. 7B, specifically, the second micro-needle template 920 has a plurality of second areas 921A, 921B, 921C and 921D and a plurality of needle-shaped structures 922, and at least one of the plurality of needle-shaped structures 922 is located in at least one of the plurality of second areas 921A, 921B, 921C and 921D. In other words, in this embodiment, the second micro-needle template 920 has a plurality of second areas 921A, 921B, 921C, and 921D. The second area 921A and the second area 921B are configured with a plurality of the needle-shaped structures 922 according to the foregoing information of the target tissue and corresponding to the first micro-needle template 910, but the second area 921C and the second area 921D are not configured with mold holes 912 and needle-shaped structures 922 on account of correspondence to the first micro-needle template 910. In addition, as mentioned above, the first micro-needle template 910 and the second micro-needle template 920 are correspondingly matched with each other in structure. The diameter and the length of the needle-shaped structures 922 correspond to the diameter and the depth of the mold holes 912 respectively. The curvature radii of the plurality of second areas 921A, 921B, 921C, and 921D correspond to the curvature radii of the plurality of first areas 911A, 911B, 911C, and 911D respectively.

Next, as shown in FIG. 8A, in the second micro-needle template configuring step S306, the second micro-needle template 920 is configured on the micro-needle material 800 and the first micro-needle template 910, such that the plurality of second areas 921A, 921B, 921C and 921D are located on the plurality of first areas 911A, 911B, 911C and 911D correspondingly, the needle-shaped structures 922 are inserted into the plurality of mold holes 912 correspondingly, and the micro-needle material 800 is located between the first micro-needle template 910 and the second micro-needle template 920.

It should be noted that before the second micro-needle template configuring step S306 is performed, the micro-needle material protection layer may also be formed, and then the second micro-needle template 920 is configured. Alternatively, before the second micro-needle template configuring step S306 is performed, the protection layer may be formed on the second micro-needle template 920 first, and then the second micro-needle template 920 may be configured. In addition, when the second micro-needle template 920 is subsequently removed, due to relatively high adhesion of the protection layer to a micro-needle semi-product 850, the protection layer is also removed when the second micro-needle template 920 is removed. Therefore, an active substance may also be added after another protection layer is formed on the micro-needle semi-product, such that the release rate of the active material can be controlled according to different usage requirements.

Next, as shown in FIG. 8B, in the micro-needle material solidifying step S307, the micro-needle material 800 is solidified to form the micro-needle semi-product 850. The micro-needle semi-product 850 has a plurality of micro-needle bodies 851, and each micro-needle body 851 has a hole 852. In other words, in the foregoing embodiment, after the micro-needle device is made, the hole is formed in the micro-needle device through a precise machining mode or a micro-electromechanical systems (MEMS) machining, and the like and filled with the active substance. In this embodiment, the second micro-needle template 920 and the first micro-needle template 910 are superimposed during a micro-needle making process by utilizing a precision machining mode or a micro-electromechanical systems (MEMS) machining, and the like, such that the needle-shaped structures 922 of the second micro-needle template 920 are inserted into the mold hole 912 of the first micro-needle template 910, and then holes are made through a molding technology. Therefore, when the micro-needle semi-product 850 is obtained, the micro-needle body 851 thereof can have the hole 852, such that in the subsequent step, the active substance 802 can directly fill therein. A solidifying mode of the micro-needle material 800 has been described above, and will not be repeated any more.

In addition, in this embodiment, the hole 852 of the micro-needle body 851 is a through hole, such that the active substance 802 can be quickly released when the body is subsequently applied to a user. In some embodiments, the hole 852 of the micro-needle body 851 may also be a closed groove, such that the micro-needle body 851 needs to be firstly dissolved in the body of the user when subsequently applied to the user, and then the active substance 802 will be released. Or, in some embodiments, more than one hole 852 of the micro-needle body 851 is formed. In this way, the hole 852 of the micro-needle body 851 can be designed to adjust the release rate of the active substance 802 according to different active substances 802 and different usage requirements.

Next, in the second micro-needle template removing step S308, the second micro-needle template 920 is removed. Next, in the active substance adding step S309, as shown in FIG. 8C, the active substance 802 is added to the micro-needle semi-product 850, such that the active substance 802 enters the holes 852. As mentioned above, in the step, the active substance 802 fills the formed hole 852. An example of the active substance 802 has been described above, and will not be repeated any more.

Finally, as shown in FIG. 8D, in the micro-needle device obtaining step S310, the first micro-needle template 910 is removed, and the micro-needle semi-product 850 is solidified to obtain a micro-needle device 860. Therefore, the micro-needle device 860 with a needle body of a syringe type micro-needle can be made.

In one or more embodiments, in the active substance adding step S309, in addition to adding the active substance 802, as mentioned above, other excipients or stabilizers may also be added such that the active substance 802 can be appropriately configured in the hole 852 of the micro-needle device 860. Or, in one or more embodiments, a macromolecular material may be added to form micelles to clad the active substance 802, and then is configured in the hole 852 of the micro-needle device 860 so as to protect the active substance 802 and even control release of the active substance 802.

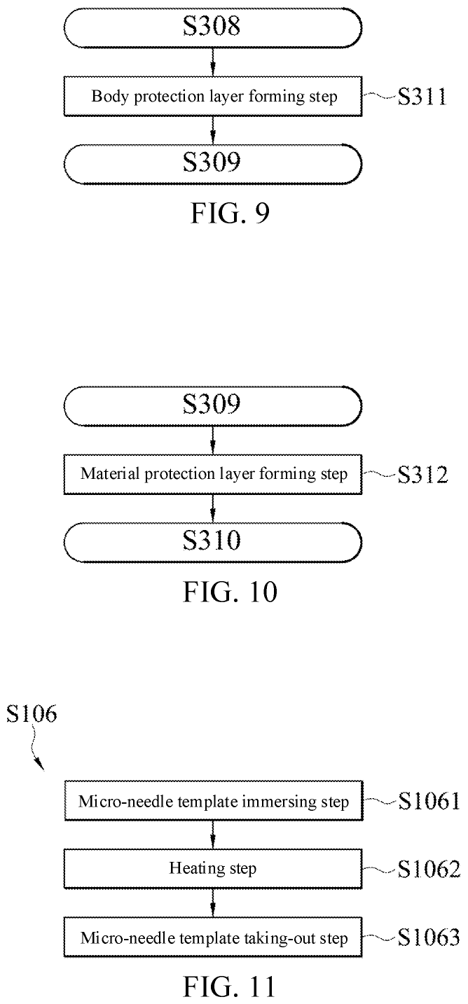

Referring to FIG. 9. FIG. 9 illustrates a partial step flowchart of a manufacturing method for a micro-needle device of Embodiment 4 of the instant disclosure. As shown in FIG. 9, in some embodiments, after a second micro-needle template removing step S308, a body protection layer forming step S311 may be performed first: a body protection layer is formed on a micro-needle semi-product, such that the body protection layer fills the holes. In some embodiments, the body protection layer is used to protect an active substance from direct contact with a micro-needle device. In some embodiments, the body protection layer may also have a function of natural degradation, thereby controlling release of the active substance. A formation method of the body protection layer is substantially the same as that of the template protection layer, except that materials and temperature conditions used may be different based on functions to be achieved by the body protection layer. Specifically, in this embodiment, a material for forming the body protection layer is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan. In other words, the body protection layer may not be removed (that is, the body protection layer and the micro-needle semi-product may be made from the same material). A preparation temperature of the body protection layer is 50° C. to 90° C., and time is about 1 to 3 hours. On the other hand, a formation temperature of the body protection layer is 50° C. to 90° C. or 0° C. to −196° C., and time is about 1 to 6 hours.

Referring to FIG. 10. FIG. 10 illustrates a partial step flowchart of a manufacturing method for a micro-needle device of Embodiment 5 of the instant disclosure. As shown in FIG. 10, in some embodiments, after an active substance adding step S309, a material protection layer forming step S312 may be performed first: a material protection layer is formed on an active substance. The material protection layer is used to protect the active substance from contact with the outside and reaction, thereby affecting the effect of the active substance. A formation method of the material protection layer is substantially the same as that of the template protection layer, except that materials and temperature conditions used may be different based on functions to be achieved by the material protection layer. Specifically, in this embodiment, a material for forming the material protection layer is selected from a group consisting of polysaccharide, poly(vinyl alcohol) (PVA), poly(lactic-co-glycolic acid) (PLGA), poly(lactic acid) (PLA), poly(glycolic acid) (PGA), carboxymethyl cellulose (CMC), chitosan, polycaprolactone (PCL), poly(dioxacyclohexane) (PDO), poly(p-dioxanone) (PPDO), poly(l-lactic acid) (PLLA), poly(propylene carbonate) (PPC), poly(dioxanone) (PDS), poly(trimethylene carbonate) (PTMC), polyvinylpyrrolidone (PVP), gelatine, trehalose, xanthan gum, locust bean gum, carrageenan, pectin, inulin, glucose, dextran, maltose and pullulan. In this embodiment, the material protection layer and a template protection layer may be made from the same material. A preparation temperature of the material protection layer is 50° C. to 90° C., and time is about 1 to 3 hours. On the other hand, a formation temperature of the material protection layer is 50° C. to 90° C. or 0° C. to −196° C., and the formation time is about 1 to 6 hours.

Referring to FIG. 11. FIG. 11 illustrates a detailed flowchart of a template protection layer forming step of one embodiment of the instant disclosure. As shown in FIG. 11, the foregoing template protection layer forming step S106 further includes: a micro-needle template immersing step S1061: immersing a micro-needle template in a protection layer solution; a heating step S1062: heating the micro-needle template and the protection layer solution to a temperature ranging from 50° C. to 90° C. for operation time to form the template protection layer on the micro-needle template, where the operation time is 2 to 5 hours; and a micro-needle template taking-out step S1063: taking the micro-needle template with the template protection layer out of the protection layer solution. Specifically, after being immersed in the protection layer solution, the micro-needle template may be allowed to stand for a period of time until bubbles disappear before heating, and the bubbles generated during the heating process may also be removed with a tool to keep the structural integrity of the template protection layer.

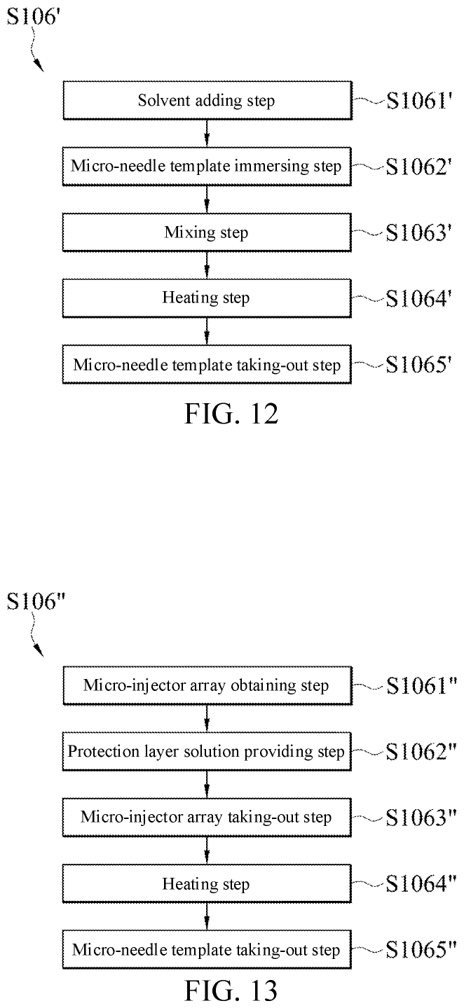

Referring to FIG. 12. FIG. 12 illustrates a detailed flowchart of a template protection layer forming step of another embodiment of the instant disclosure. As shown in FIG. 12, the foregoing template protection layer forming step S106′ further includes: a solvent adding step S1061′: adding a solvent to a micro-needle template; a micro-needle immersing step S1062′: immersing a micro-needle template in a protection solution tank, where the protection solution tank contains the protection layer solution; a mixing step S1063′: mixing the solvent and the protection layer solution; a heating step S1064′: heating the protection solution tank to a temperature ranging from 50° C. to 90° C. for operation time to form the template protection layer on the micro-needle template, where the operation time is 2 to 5 hours; and a micro-needle template taking-out step S1065′: taking the micro-needle template with the template protection layer out of the protection solution tank. Specifically, in this embodiment, the solvent (such as water) fills holes in the micro-needle template and drives the bubbles out. Then, the micro-needle template is immersed together with the solvent into the protection solution tank containing the protection layer solution, and the solvent and the protection layer solution are mixed. Therefore, the formed template protection layer can minimize residual bubbles and ensure the structural integrity of the template protection layer.

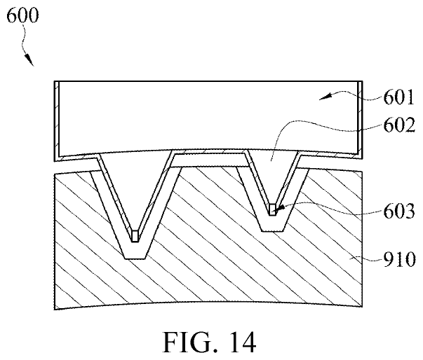

Referring to FIG. 13 and FIG. 14. FIG. 13 illustrates a detailed flowchart of a template protection layer forming step of yet another embodiment of the instant disclosure. FIG. 14 illustrates a schematic cross-sectional view of a micro-needle template matched with a micro-injector array corresponding to the embodiment shown by FIG. 13. As shown in FIG. 13 and FIG. 14, the foregoing template protection layer forming step S106″ further includes: a micro-injector array obtaining step S1061″: obtaining the micro-injector array by utilizing a three-dimensional scanning technology or optical coherence tomography; a protection layer solution providing step S1062″: providing a protection layer solution into a container, and enabling the protection layer solution to pass through the needle holes, be located in the areas and enter into the plurality of mold holes; a micro-injector array taking-out step S1063″: taking the micro-injector array out; a heating step S1064″: heating the micro-needle template and the protection layer solution to a temperature ranging from 50° C. to 90° C. for operation time to form a micro-needle protection layer on the micro-needle template, where the operation time is 2 to 5 hours; and a micro-injector template taking-out step S1065″: taking the micro-needle template is out of the protection layer solution. As shown in FIG. 14, the micro-injector array 600 has a container 601 and a plurality of injection needles 602, each injection needle 602 has a needle hole 603 for communicating with the container 601, and the size of the plurality of injection needles 602 corresponds to the diameter and the depth of the plurality of mold holes. Specifically, in this embodiment, because the micro-injector array 600 is obtained according to the three-dimensional scanning technology or the optical coherence tomography, and the micro-needle template is also obtained according to such technologies, the size of the injection needle 602 corresponds to the diameter and the depth of the mold hole. Therefore, the micro-syringe array can correspondingly insert the injection needle thereof into the mold hole of the micro-needle template, so that air in the mold hole is discharged, and generation of bubbles is reduced. Then, the protection layer solution is placed in the mold hole through the injection needle 602.

It should be noted that a detailed manufacturing process of the above template protection layer is described in Embodiment 2, but it is not limited to this. A manufacturing process of the template protection layer may also be applicable to templates described in other embodiments of the instant disclosure. The protection layer may also be used as a template protection layer of the first micro-needle template and the second micro-needle template, which is not repeated any more. In addition, the above manufacturing process of the template protection layer is used to reduce air bubbles and effect on a finished product during a molding process, so that it may also be applicable to filling of the molding material or the active substance, as well as production of other foregoing protection layers.

In addition, although the micro-needle template/the micro-needle device/the micro-injector array shown in the drawings have curvature, as mentioned above, the curvature radius of the micro-needle template/the micro-needle device/the micro-injector array is only illustrative. In some embodiments, the micro-needle template/the micro-needle device/the micro-injector array may not have the curvature radius but be arranged in a plane.

Based on the foregoing description, micro-needle devices of Examples 1 to 6 are manufactured according to the foregoing manufacturing method for the micro-needle device.

### Example 1

| | | | Thickness |
|---|---|---|---|
| | | | (without |
| | | Operation | micro-needle |
| | Composition | time | length) |
| Molding | Poly(vinyl alcohol), | 2 to 8 hours | 1 to 5 mm |
| substance | trehalose, xanthan | | |
| | gum and locust | | |
| | bean gum | | |
| Active | Hyaluronic acid | 2 to 3 hours | 2 to 3 mm |
| substance | | | |
| Material | Poly(vinyl alcohol) | 2 to 5 hours | 0.1 to 2 mm |
| protection | | | |
| layer | | | |
| Body | Poly(vinyl alcohol) | 2 to 5 hours | 0.1 to 2 mm |
| protection | | | |
| layer | | | |

### Example 2

| | | | Thickness |
|---|---|---|---|
| | | | (without |
| | | Operation | micro-needle |
| | Composition | time | length) |
| Molding | Poly(vinyl alcohol), | 2 to 8 hours | 1 to 5 mm |
| substance | trehalose, xanthan | | |
| | gum and locust | | |
| | bean gum | | |
| Active | Collagen | 2 to 3 hours | 2 to 3 mm |
| substance | | | |
| Material | Poly(vinyl alcohol) | 2 to 5 hours | 0.1 to 2 mm |
| protection | | | |
| layer | | | |
| Body | Poly(vinyl alcohol) | 2 to 5 hours | 0.1 to 2 mm |
| protection | | | |
| layer | | | |

### Example 3

| | | | Thickness |
|---|---|---|---|
| | | | (without |
| | | Operation | micro-needle |
| | Composition | time | length) |
| Molding | Poly(vinyl alcohol), | 1 to 5 hours | 2 to 5 mm |
| substance | sorbitol, citric acid | | |
| | and sodium citrate | | |
| Active | Hyaluronic acid | 2 to 3 hours | 2 to 3 mm |
| substance | | | |
| Material | Poly(vinyl alcohol) | 2 to 5 hours | 0.1 to 2 mm |
| protection | | | |
| layer | | | |
| Body | Poly(vinyl alcohol) | 2 to 5 hours | 0.1 to 2 mm |
| protection | | | |
| layer | | | |

### Example 4

| | | | Thickness |
|---|---|---|---|
| | | | (without |
| | | Operation | micro-needle |
| | Composition | time | length) |
| Molding | Poly(vinyl alcohol), | 1 to 5 hours | 2 to 5 mm |
| substance | sorbitol, citric acid | | |
| | and sodium citrate | | |
| Active | Collagen | 2 to 3 hours | 2 to 3 mm |
| substance | | | |
| Material | Poly(vinyl alcohol) | 2 to 5 hours | 0.1 to 2 mm |
| protection | | | |
| layer | | | |
| Body | Poly(vinyl alcohol) | 2 to 5 hours | 0.1 to 2 mm |
| protection | | | |
| layer | | | |

It can be confirmed from the above examples that the foregoing manufacturing method for the micro-needle device can adjust the composition of the molding substance and the active substance, and corresponds to different operation times and temperatures, thereby meeting different requirements of a user.

Moreover, based on the foregoing description, a micro-needle device is manufactured according to the foregoing manufacturing method for the micro-needle device for pasting testing.

### Example 5

A micro-needle device with the molding substance of the poly(vinyl alcohol), the sorbitol, the citric acid and the sodium citrate is pasted to pigskin. Needle-shaped surface textures of the micro-needle begin to disappear after 30 minutes, and the and needle-shaped the surface textures of the micro-needle disappear completely in 60 minutes after pasting. A needle-shaped contour of the micro-needle still exists but a needle body begins to soften in 150 minutes after pasting.

### Example 6

A micro-needle device with the molding substance of poly(vinyl alcohol), carboxymethyl cellulose, and polyvinylpyrrolidone is pasted to pigskin. A needle tip of the micro-needle begins to dissolve after 30 minutes, about 30% of the needle shape of the micro-needle is dissolved 60 minutes after pasting, and the needle shape of the micro-needle is almost completely dissolved 180 minutes after pasting.

It can be confirmed from the above examples that the micro-needle device manufactured by the foregoing manufacturing method for the micro-needle device can adjust composition of the molding substance and the active substance to achieve different degrees of release efficiency, thereby meeting different requirements of a user.

In summary, according to one or more embodiments of the instant disclosure, a micro-needle device with a syringe or mixed type needle body can be manufactured according to different usage requirements, and a high-specificity micro-needle device product can be made corresponding to specific skin surface curvature information and inner tissue distribution information of a user. In addition, in some embodiments, the skin condition of the user may also be understood according to the inner tissue distribution information, then in the active substance adding step, different positions of the micro-needle device have different contents of active substances, so that the provision efficiency of the active substance is optimized. In still other embodiments, air bubbles can be reduced during molding, thereby ensuring the integrity of the protection layer/the micro-needle device.

What is claimed is:

1. A manufacturing method for a micro-needle device, comprising:
a target tissue basic information obtaining step: obtaining skin surface curvature information of a target tissue and inner tissue distribution information of the target tissue, wherein the inner tissue distribution information is obtained by applying optical coherence tomography;
a micro-needle template obtaining step: obtaining a micro-needle template according to the skin surface curvature information and the inner tissue distribution information, wherein the micro-needle template has a plurality of areas and a plurality of mold holes, at least one of the plurality of mold holes is located in at least one of the plurality of areas, at least one of the diameter and the depth of the plurality of mold holes is determined by the inner tissue distribution information, and the curvature radius of the plurality of areas is determined by the skin surface curvature information;
a micro-needle material adding step: adding a micro-needle material to the micro-needle template, such that the micro-needle material is located on the plurality of areas and fills the plurality of mold holes, wherein the micro-needle material comprises a molding substance;
a micro-needle semi-product obtaining step: solidifying the micro-needle material to form a micro-needle semi-product; and
a micro-needle device obtaining step: removing the micro-needle template to obtain the micro-needle device.

2. The manufacturing method for the micro-needle device according to claim 1, wherein the micro-needle semi-product obtaining step is performed under a temperature ranging from 0° C. to −196° C., and the micro-needle material further comprises an active substance; and the micro-needle device obtaining step is to solidify the micro-needle semi-product under a temperature ranging from 50° C. to 90° C. to obtain the micro-needle device.

3. The manufacturing method for the micro-needle device according to claim 1, wherein the micro-needle semi-product obtaining step is performed under a temperature ranging from 50° C. to 90° C.

4. The manufacturing method for the micro-needle device according to claim 2, wherein the molding substance is selected from a group consisting of polysaccharide, poly(vinyl alcohol), poly(lactic-co-glycolic acid), poly(lactic acid), poly(glycolic acid), carboxymethyl cellulose, chitosan, polycaprolactone, poly(dioxacyclohexane), poly(p-dioxanone), poly(l-lactic acid), poly(propylene carbonate), poly(dioxanone), poly(trimethylene carbonate), polyvinylpyrrolidone, gelatine, trehalose, xanthan gum, locust bean gum, carrageenan, pectin, inulin, glucose, dextran, maltose and pullulan.

5. The manufacturing method for the micro-needle device according to claim 3, wherein the molding substance is selected from a group consisting of polysaccharide, poly(vinyl alcohol), poly(lactic-co-glycolic acid), poly(lactic acid), poly(glycolic acid), carboxymethyl cellulose, chitosan, polycaprolactone, poly(dioxacyclohexane), poly(p-dioxanone), poly(l-lactic acid), poly(propylene carbonate), poly(dioxanone), poly(trimethylene carbonate), polyvinylpyrrolidone, gelatine, trehalose, xanthan gum, locust bean gum, carrageenan, pectin, inulin, glucose, dextran, maltose and pullulan.

6. The manufacturing method for the micro-needle device according to claim 1, wherein the micro-needle semi-product obtaining step is performed under a room temperature, the micro-needle material further comprises an active substance, and the molding substance is collagen or hyaluronic acid; and the micro-needle device obtaining step is to solidify the micro-needle semi-product under a temperature ranging from 50° C. to 90° C. to obtain the micro-needle device.

7. The manufacturing method for the micro-needle device according to claim 1, before the micro-needle material adding step, further comprising a template protection layer forming step: forming a template protection layer on the micro-needle template under a temperature ranging from 50° C. to 90° C., such that the template protection layer is located on the plurality of areas and fills the plurality of the mold holes, wherein the micro-needle material is located on the plurality of areas and on the template protection layer, the template protection layer is selected from a group consisting of polysaccharide, poly(vinyl alcohol), poly(lactic-co-glycolic acid), poly(lactic acid), poly(glycolic acid), carboxymethyl cellulose, chitosan, polycaprolactone, poly(dioxacyclohexane), poly(p-dioxanone), poly(l-lactic acid), poly(propylene carbonate), poly(dioxanone), poly(trimethylene carbonate), polyvinylpyrrolidone, gelatine, trehalose, xanthan gum, locust bean gum, carrageenan, pectin, inulin, glucose, dextran, maltose and pullulan, and the micro-needle material further comprises an active substance; and the micro-needle device obtaining step is to remove the micro-needle template and the template protection layer to obtain the micro-needle device.

8. The manufacturing method for the micro-needle device according to claim 7, wherein the micro-needle semi-product obtaining step is performed under a room temperature or under a temperature ranging from 0° C. to −196° C.; and the micro-needle device obtaining step is to solidify the micro-needle semi-product under a temperature ranging from 50° C. to 90° C. to obtain the micro-needle device.

9. The manufacturing method for the micro-needle device according to claim 1, wherein the micro-needle semi-product obtaining step is performed under a temperature ranging from 0° C. to −196° C. or under a temperature ranging from 50° C. to 90° C.

10. The manufacturing method for the micro-needle device according to claim 7, wherein the micro-needle semi-product obtaining step is performed under a temperature ranging from 50° C. to 90° C., and the molding substance is collagen or hyaluronic acid; and the micro-needle device obtaining step is to solidify the micro-needle semi-product under a temperature ranging from 50° C. to 90° C. to obtain the micro-needle device.

11. The manufacturing method for the micro-needle device according to claim 7, wherein the micro-needle semi-product obtaining step is performed under a temperature ranging from 50° C. to 90° C., and the molding substance is the polysaccharide; and the micro-needle device obtaining step is to solidify the micro-needle semi-product under a temperature ranging from 0° C. to −196° C. or from 50° C. to 90° C. to obtain the micro-needle device.

12. The manufacturing method for the micro-needle device according to claim 7, wherein the template protection layer forming step further comprises:
immersing the micro-needle template in a protection layer solution;
heating the micro-needle template and the protection layer solution to a temperature ranging from 50° C. to 90° C. to form the template protection layer on the micro-needle template; and
taking the micro-needle template with the template protection layer out of the protection layer solution.

13. The manufacturing method for the micro-needle device according to claim 7, wherein the template protection layer forming step further comprises:
adding a solvent to the micro-needle template;
immersing the micro-needle template in a protection solution tank, wherein the protection solution tank contains a protection layer solution;
mixing the solvent and the protection layer solution;
heating the protection solution tank to a temperature ranging from 50° C. to 90° C. to form the template protection layer on the micro-needle template; and
taking the micro-needle template with the template protection layer out of the protection solution tank.

14. The manufacturing method for the micro-needle device according to claim 7, wherein the template protection layer forming step further comprises:
obtaining a micro-injector array by utilizing a three-dimensional scanning technology or the optical coherence tomography, wherein the micro-injector array has a container and a plurality of injection needles, each of the plurality of injection needle has a needle hole for communicating with the container, and the size of the plurality of injection needles corresponds to the diameter and the depth of the plurality of mold holes;
providing a protection layer solution into the container, and enabling the protection layer solution to pass through the needle holes, be located in the plurality of areas and enter into the plurality of mold holes;
taking the micro-injector array out;
heating the micro-needle template and the protection layer solution to a temperature ranging from 50° C. to 90° C. to form a micro-needle protection layer on the micro-needle template; and
taking the micro-needle template out of the protection layer solution.

15. A manufacturing method for a micro-needle device, comprising:
a target tissue basic information obtaining step: obtaining skin surface curvature information of a target tissue and inner tissue distribution information of the target tissue, wherein the inner tissue distribution information is obtained by applying optical coherence tomography;
a first micro-needle template obtaining step: obtaining a first micro-needle template according to the skin surface curvature information and the inner tissue distribution information, wherein the first micro-needle template has a plurality of first areas and a plurality of mold holes, at least one of the plurality of mold holes is located in at least one of the plurality of first areas, at least one of the diameter and the depth of the plurality of mold holes is determined by the inner tissue distribution information, and the curvature radius of the plurality of first areas is determined by the skin surface curvature information;
a template protection layer forming step: forming a template protection layer on the first micro-needle template, such that the template protection layer is located on the plurality of first areas and fills the plurality of mold holes;
a micro-needle material adding step: adding a micro-needle material to the template protection layer, such that the micro-needle material is located on the plurality of areas and fills the plurality of mold holes, wherein the micro-needle material comprises a molding sub stance;
a second micro-needle template obtaining step: obtaining a second micro-needle template according to the skin surface curvature information and the inner tissue distribution information, wherein the second micro-needle template has a plurality of second areas and a plurality of needle-shaped structures, at least one of the plurality of needle-shaped structures is located in at least one of the plurality of second areas, the diameter and the length of the plurality of needle-shaped structures correspond to the diameter and the depth of the plurality of mold holes respectively, and a curvature radius of the plurality of second areas corresponds to a curvature radius of the plurality of first areas;
a second micro-needle template configuring step: configuring the second micro-needle template on the micro-needle material and the first micro-needle template, such that the plurality of second areas are located on the plurality of first areas correspondingly, the plurality of needle structures are inserted into the plurality of mold holes correspondingly, and the micro-needle material is located between the first micro-needle template and the second micro-needle template;
a micro-needle material solidifying step: solidifying the micro-needle material to form a micro-needle semi-product, wherein the micro-needle semi-product has a plurality of micro-needle bodies, and each of the plurality of micro-needle body has a hole;
a second micro-needle template removing step: removing the second micro-needle template;
an active substance adding step: adding an active substance to the micro-needle semi-product, such that the active substance enters the holes; and
a micro-needle device obtaining step: removing the first micro-needle template and solidifying the micro-needle semi-product to obtain the micro-needle device.

16. The manufacturing method for the micro-needle device according to claim 15, wherein the template protection layer forming step further comprises:
immersing the first micro-needle template in a protection layer solution;
heating the first micro-needle template and the protection layer solution to a temperature ranging from 50° C. to 90° C. to form the template protection layer on the first micro-needle template; and
taking the first micro-needle template with the template protection layer out of the protection layer solution.

17. The manufacturing method for the micro-needle device according to claim 15, wherein the template protection layer forming step further comprises:
adding a solvent to the first micro-needle template;
immersing the first micro-needle template in a protection solution tank, wherein the protection solution tank contains a protection layer solution;
mixing the solvent and the protection layer solution;
heating the protection solution tank to a temperature ranging from 50° C. to 90° C. to form the template protection layer on the first micro-needle template; and
taking the first micro-needle template with the template protection layer out of the protection solution tank.

18. The manufacturing method for the micro-needle device according to claim 15, wherein the template protection layer forming step further comprises:
obtaining a micro-injector array by utilizing a three-dimensional scanning technology or the optical coherence tomography, wherein the micro-injector array has a container and a plurality of injection needles, each of the plurality of injection needle has a needle hole for communicating with the container, and the size of the plurality of injection needles corresponds to the diameter and the depth of the plurality of mold holes;
providing a protection layer solution into the container, and enabling the protection layer solution to pass through the needle holes, be located in the plurality of first areas and enter into the plurality of mold holes;
taking the micro-injector array out;
heating the first micro-needle template and the protection layer solution to a temperature ranging from 50° C. to 90° C. to form the micro-needle protection layer on the first micro-needle template; and
taking the first micro-needle template out of the protection layer solution.
